(19) United States
(12) Patent Application Publication
(10) Pub. No.: US 2024/0299015 A1
(43) Pub. Date: Sep. 12, 2024

(54) BIOPSY SYSTEM, GUIDE DEVICE, AND BIOPSY METHOD

(71) Applicant: FUJIFILM Corporation, Tokyo (JP)

(72) Inventors: Lisako NOBUYAMA, Kanagawa (JP); Hisatsugu HORIUCHI, Kanagawa (JP); Sayaka SAITO, Kanagawa (JP)

(73) Assignee: FUJIFILM Corporation, Tokyo (JP)

(21) Appl. No.: 18/597,849

(22) Filed: Mar. 6, 2024

(30) Foreign Application Priority Data
Mar. 10, 2023 (JP) 2023-038162

(51) Int. Cl. A61B 10/02; A61B 17/34

(52) U.S. Cl. CPC A61B 10/0233; A61B 17/3403; A61B 2017/3413

(57) ABSTRACT

A biopsy system performs a biopsy of a breast while capturing an ultrasound image with an ultrasonography apparatus, and includes: a compression member that has an opening portion into which a biopsy needle and an ultrasound probe used for capturing the ultrasound image are inserted and that compresses the breast; at least one processor that performs guide processing for guiding a target insertion position and a target insertion angle of the biopsy needle inserted into the breast and a target scanning position of the ultrasound probe; and an acoustic matching body that is provided between the ultrasound probe and the breast in a non-contact state with the biopsy needle.

### CROSS REFERENCE TO RELATED APPLICATIONS

This application claims priority from Japanese Application No. 2023-038162, filed on Mar. 10, 2023, the entire disclosure of which is incorporated herein by reference.

### BACKGROUND

### 1. Technical Field

The present disclosure relates to a biopsy system, a guide device, and a biopsy method.

### 2. Related Art

A technology is known in which a breast biopsy is performed while observing a tissue around a puncture needle under ultrasound guidance (see, for example, JP2019-13788A).

Generally, in the related art, in ultrasound images, the above technology is performed after applying an acoustic matching body for acoustic impedance matching to a subject.

In such a case, an ultrasound probe and a biopsy needle are approached from a separate location to avoid contamination of the biopsy needle by the acoustic matching body. However, in order to obtain better ultrasound images, it is desirable that the ultrasound probe is applied to a location close to an imaging target, and in order to reduce the invasive range of the biopsy needle, it is also desirable that the puncture position of the biopsy needle is close to a biopsy target part.

### SUMMARY

The present disclosure has been made in consideration of the above-described circumstances, and an object of the present disclosure is to provide a biopsy system, a guide device, and a biopsy method that can perform a biopsy of a breast with reference to a high-quality ultrasound image without a need to apply any one of an ultrasound probe or a biopsy needle to a position far from a biopsy target in a state in which contamination of the biopsy needle is prevented.

In order to achieve the above object, according to a first aspect of the present disclosure, there is provided a biopsy system that performs a biopsy of a breast while capturing an ultrasound image with an ultrasonography apparatus, the biopsy system comprising: a compression member that has an opening portion into which a biopsy needle and an ultrasound probe used for capturing the ultrasound image are inserted and that compresses the breast; at least one processor configured to perform guide processing for guiding a target insertion position and a target insertion angle of the biopsy needle inserted into the breast and a target scanning position of the ultrasound probe; and an acoustic matching body that is provided between the ultrasound probe and the breast in a non-contact state with the biopsy needle.

According to a second aspect of the present disclosure, in the biopsy system according to the first aspect, the acoustic matching body may be mounted on the ultrasound probe.

According to a third aspect of the present disclosure, in the biopsy system according to the first aspect, the processor may be configured to: acquire a current insertion position and a current insertion angle of the biopsy needle inserted into the breast; acquire a current scanning position of the ultrasound probe; and perform the guide processing based on the current insertion position and the current insertion angle of the biopsy needle, the current scanning position of the ultrasound probe, a current position of a distal end of the biopsy needle, the target insertion position and the target insertion angle of the biopsy needle, and the target scanning position of the ultrasound probe.

According to a fourth aspect of the present disclosure, in the biopsy system according to the first aspect, the processor may be configured to, as the guide processing: acquire at least one of a radiation image of the breast or the ultrasound image captured by the ultrasonography apparatus; derive the target insertion position and the target insertion angle of the biopsy needle based on a region of interest in the radiation image of the breast or a region of interest in the ultrasound image of the breast, and present the derived target insertion position and target insertion angle of the biopsy needle; and derive the target scanning position of the ultrasound probe that is in a non-contact position with the biopsy needle based on the derived target insertion position and target insertion angle of the biopsy needle, and present the derived target scanning position of the ultrasound probe.

According to a fifth aspect of the present disclosure, in the biopsy system according to the fourth aspect, the processor may be configured to: in a case in which a target is calcification, derive the target insertion position and the target insertion angle of the biopsy needle based on the region of interest in the radiation image; and in a case in which the target is a tumor, derive the target insertion position and the target insertion angle of the biopsy needle based on the region of interest in the ultrasound image.

According to a sixth aspect of the present disclosure, the biopsy system according to the first aspect may further comprise a moving mechanism that moves the ultrasound probe, and the processor may be configured to present the target scanning position of the ultrasound probe to the moving mechanism, and the moving mechanism moves the ultrasound probe to the target scanning position.

According to a seventh aspect of the present disclosure, in the biopsy system according to the sixth aspect, the moving mechanism may enable capturing of ultrasound images in a major axis direction and a minor axis direction of the biopsy needle by rotating the ultrasound probe with respect to the biopsy needle.

According to an eighth aspect of the present disclosure, in the biopsy system according to the first aspect, the processor may be configured to perform guide processing for guiding a scanning position of the ultrasound probe to a position where a final destination of the biopsy needle based on the derived target insertion position and target insertion angle of the biopsy needle is able to be imaged.

According to a ninth aspect of the present disclosure, the biopsy system according to the first aspect may further comprise a disposition mechanism for disposing the biopsy needle, the processor may be configured to present the target insertion position and the target insertion angle of the biopsy needle to the disposition mechanism, and the disposition mechanism may dispose the biopsy needle according to the target scanning position and the target insertion angle.

According to a tenth aspect of the present disclosure, in the biopsy system according to the first aspect, the processor may be configured to: determine whether or not there is a likelihood that the acoustic matching body and the biopsy needle come into contact with each other; and give a warning in a case in which there is a likelihood of contact.

According to an eleventh aspect of the present disclosure, the biopsy system according to the first aspect may further comprise an image processing apparatus configured to: acquire a plurality of ultrasound images captured by the ultrasonography apparatus; and generate a three-dimensional ultrasound image from the plurality of ultrasound images.

According to a twelfth aspect of the present disclosure, in the biopsy system according to the first aspect, the acoustic matching body may be in a form of a gel, and the processor may be configured to: acquire at least one of a radiation image of the breast or the ultrasound image captured by the ultrasonography apparatus; derive the target insertion position and the target insertion angle of the biopsy needle based on a region of interest in the radiation image of the breast or a region of interest in the ultrasound image of the breast; and further perform guide processing for guiding a disposition position of the acoustic matching body to a disposition position where the acoustic matching body does not come into contact with the biopsy needle based on the derived target insertion position and target insertion angle of the biopsy needle.

Further, in order to achieve the above object, according to a thirteenth aspect of the present disclosure, there is provided a guide device used in a biopsy system that performs a biopsy of a breast while capturing an ultrasound image with an ultrasonography apparatus, the guide device comprising: at least one processor, in which the processor is configured to perform guide processing for guiding a target insertion position and a target insertion angle of a biopsy needle inserted into the breast and a target scanning position of an ultrasound probe.

In addition, in order to achieve the object, according to a fourteenth aspect of the present disclosure, there is provided a biopsy method using a biopsy system that performs a biopsy of a breast while capturing an ultrasound image with an ultrasonography apparatus, the biopsy method comprising: performing guide processing for guiding, by a processor, in a state in which the breast is compressed by a compression member having an opening portion into which a biopsy needle and an ultrasound probe used for capturing the ultrasound image are inserted, and an acoustic matching body is provided between the ultrasound probe and the breast in a non-contact state with the biopsy needle, a target insertion position and a target insertion angle of the biopsy needle inserted into the breast and a target scanning position of the ultrasound probe.

According to the aspects of the present disclosure, it is possible to perform a breast biopsy with reference to a high-quality ultrasound image without a need to apply any one of an ultrasound probe or a biopsy needle to a position far from a biopsy target in a state in which contamination of the biopsy needle is prevented.

### BRIEF DESCRIPTION OF THE DRAWINGS

FIG. 1 is a configuration diagram schematically showing an example of the overall configuration of a biopsy system according to a first embodiment.

FIG. 2 is a side view showing an example of the appearance of a medical imaging apparatus of the first embodiment.

FIG. 3 is a block diagram showing an example of a compression member of the first embodiment.

FIG. 4 is a block diagram showing an example of a moving mechanism of an ultrasound probe of the first embodiment.

FIG. 5A is a diagram for describing a rotation of an ultrasound probe.

FIG. 5B is a diagram for describing a rotation of an ultrasound probe.

FIG. 6 is a block diagram showing an example of the configuration of the medical imaging apparatus and a console of the first embodiment.

FIG. 7 is a functional block diagram showing an example of the configuration of the console of the first embodiment.

FIG. 8 is a flowchart showing an example of a flow of biopsy processing executed in the console of the first embodiment.

FIG. 9 is a functional block diagram showing an example of the configuration of a console of a third embodiment.

FIG. 10 is a flowchart showing a part of an example of a flow of biopsy processing executed in a console of a fourth embodiment.

### DESCRIPTION OF EMBODIMENTS

Embodiments of the present invention will be described in detail below with reference to the drawings. Note that the present embodiment does not limit the present invention.

### First Embodiment

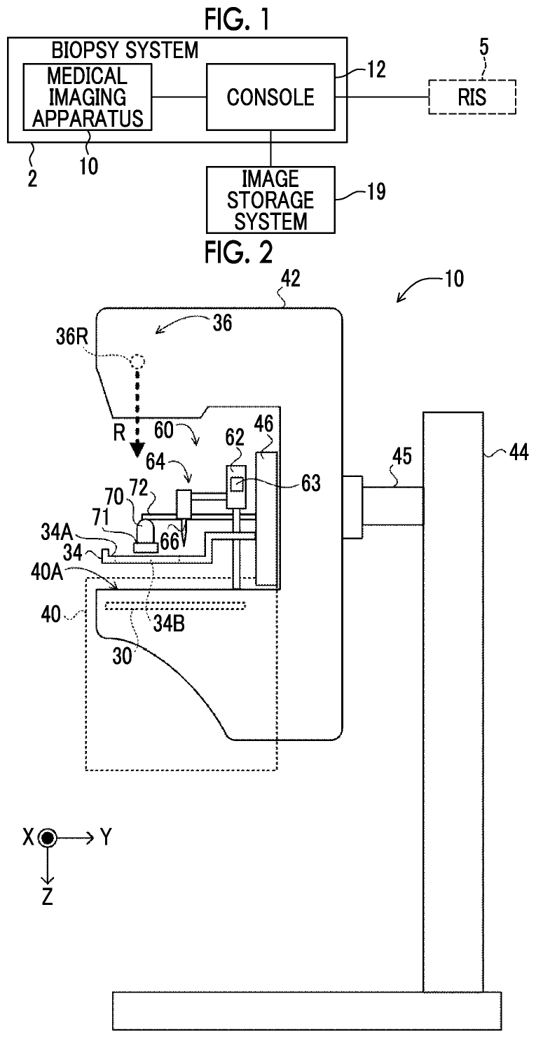

First, an example of the overall configuration of a biopsy system 2 according to the present embodiment will be schematically described. FIG. 1 is a configuration diagram schematically showing an example of the overall configuration of the biopsy system 2 according to the present embodiment. As shown in FIG. 1, the biopsy system 2 comprises a medical imaging apparatus 10 and a console 12.

The medical imaging apparatus 10 is an apparatus that is capable of capturing a radiation image and an ultrasound image of a breast of an examinee used as a subject and is also capable of taking a biopsy of the breast. For example, the medical imaging apparatus 10 of the present embodiment is an apparatus that is obtained by adding a function of performing ultrasound imaging to a mammography apparatus that can perform a biopsy.

FIG. 2 is a side view showing an example of the appearance of the medical imaging apparatus 10 of the present embodiment. In addition, FIG. 2 is a side view showing the medical imaging apparatus 10 as viewed from the right side of an examinee. As shown in FIG. 2, the medical imaging apparatus 10 comprises a radiation source 36R, a radiation detector 30, an imaging table 40 disposed between the radiation source 36R and the radiation detector 30, and a compression member 34 that compresses the breast between the compression member 34 and the imaging table 40.

The radiation detector 30 is disposed inside the imaging table 40. The radiation detector 30 detects the radiation R transmitted through the breast, which is the subject. In the medical imaging apparatus 10 of the present embodiment, in a case in which imaging is performed, the breast of the examinee is positioned on an imaging surface 40A of the imaging table 40 by a user such as a doctor or a radiology technician. For example, the imaging surface 40A and the like with which the breast of the examinee comes into contact are made of carbon or the like in terms of the transmittance and intensity of the radiation R.

The radiation detector 30 detects the radiation R transmitted through the breast of the examinee and the imaging table 40, generates a radiation image based on the detected radiation R, and outputs the generated radiation image. The type of the radiation detector 30 of the present embodiment is not particularly limited. For example, the radiation detector 30 may be an indirect conversion type radiation detector that converts the radiation R into light and converts the converted light into electric charges, or may be a direct conversion type radiation detector that directly converts the radiation R into electric charges.

The radiation source 36R is provided in a radiation emitting unit 36. As shown in FIG. 2, the radiation emitting unit 36 is provided on the arm part 42 together with the imaging table 40 and a compression unit 46. In addition, as shown in FIG. 2, the medical imaging apparatus 10 of the present embodiment comprises the arm part 42, a base 44, and a shaft part 45. The arm part 42 is held by the base 44 so as to be movable in an up-down direction (Z-axis direction). The shaft part 45 connects the arm part 42 to the base 44. Further, the arm part 42 can be relatively rotated with respect to the base 44 by a radiation source moving unit 37, using the shaft part 45 as a rotation axis. By rotating the arm part 42 relative to the base 44, the incidence angle of radiation to the breast placed on the imaging surface 40A of the imaging table 40 can be varied. In this way, the medical imaging apparatus 10 of the present embodiment is capable of so-called stereo imaging and tomosynthesis imaging.

Further, as shown in FIG. 2, the compression member 34 is attached to the compression unit 46. The compression unit 46 and the arm part 42 can be relatively rotated with respect to the base 44 separately, using the shaft part 45 as a rotation axis. In the present embodiment, gears (not shown) are provided in each of the shaft part 45, the arm part 42, and the compression unit 46. Each gear is switched between an engaged state and a disengaged state to connect each of the arm part 42 and the compression unit 46 to the shaft part 45. One or both of the arm part 42 and the compression unit 46 connected to the shaft part 45 are rotated integrally with the shaft part 45.

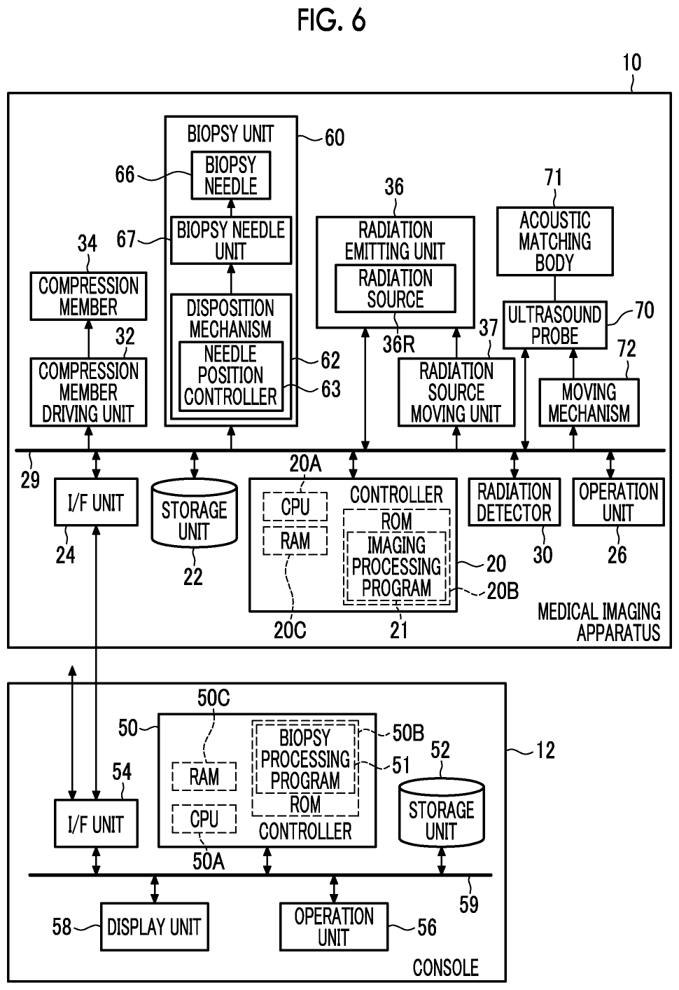

The compression member 34 of the present embodiment is a plate-shaped member, and is moved in the up-down direction (Z-axis direction) by a compression member driving unit 32 (see FIG. 6) provided in the compression unit 46 to compress the breast of the examinee between the compression member 34 and the imaging table 40.

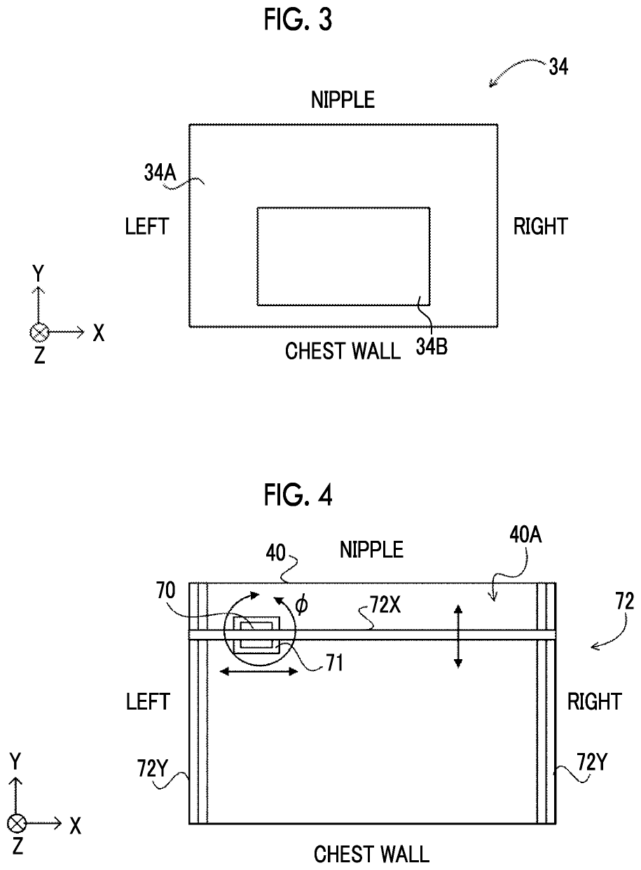

It is preferable that the compression member 34 is optically transparent in order to check positioning or a compression state in the compression of the breast. In addition, the compression member 34 is made of a material having high transmittance for the radiation R. Further, the compression member 34 of the present embodiment is a compression member for a biopsy, and as shown in FIG. 3, the compression member 34 has an opening portion 34B in a bottom portion 34A that contacts the breast in the case of compressing the breast. A biopsy needle 66 and an ultrasound probe 70 can be inserted into the opening portion 34B.

In addition, as shown in FIG. 2, the medical imaging apparatus 10 of the present embodiment comprises a biopsy unit 60. The biopsy unit 60 includes a disposition mechanism 62, the biopsy needle 66, and a biopsy needle unit 67. The biopsy needle 66 is provided in the biopsy needle unit 67, and is used to puncture the breast and collect tissue. The disposition mechanism 62 has a function of moving the biopsy needle 66 in X, Y, and Z directions to dispose the biopsy needle 66 at a target insertion position. In the present embodiment, the X direction is a left-right direction of the examinee, and the Y direction is a front-rear direction of the examinee. Further, the Z direction is a direction perpendicular to the XY plane, and is an up-down direction of the examinee. Therefore, hereinafter, the X direction will be referred to as the “left-right direction”, the Y direction will be referred to as the “front-rear direction”, and the Z direction will be referred to as the “up-down direction”.

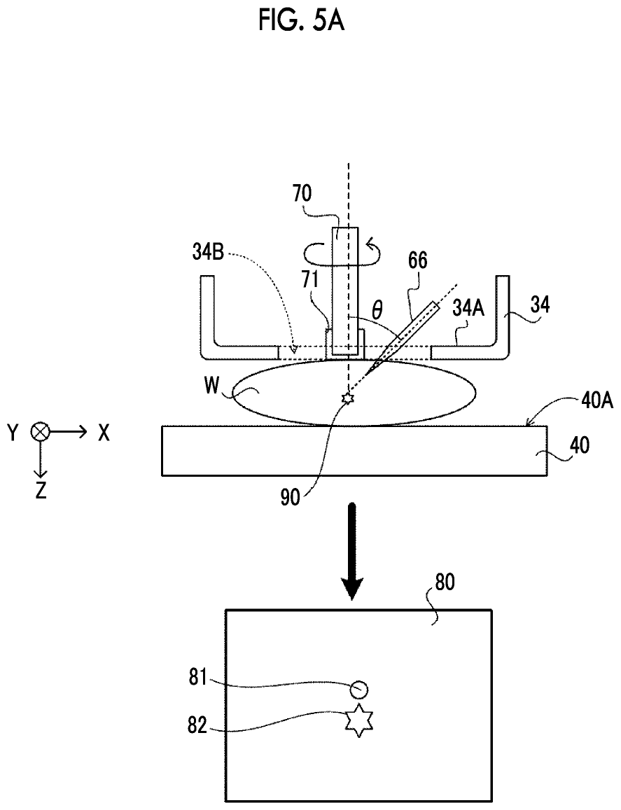

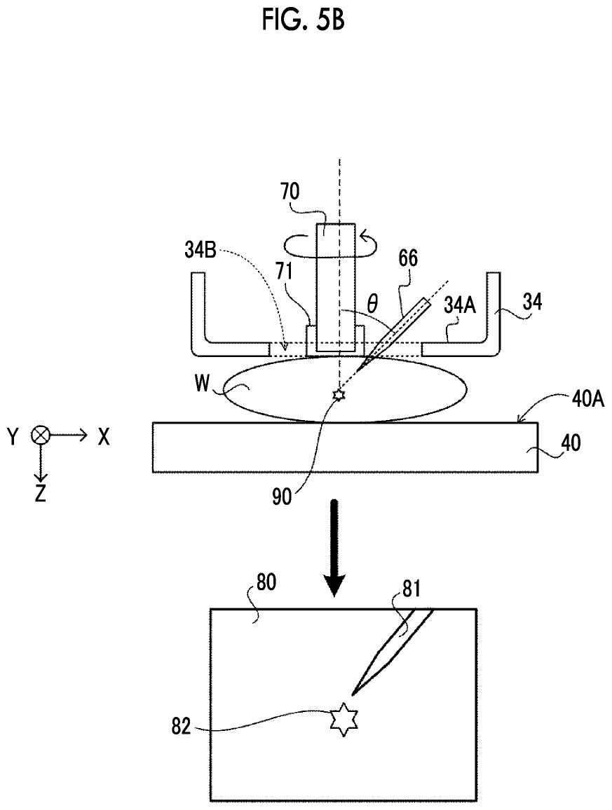

The disposition mechanism 62 includes a needle position controller 63, and a position of a distal end of the biopsy needle 66 is controlled by the needle position controller 63. Upon receiving the position information of a target, the needle position controller 63 moves a position of the distal end of the biopsy needle 66 to a target insertion position corresponding to the position of the target, and tilts the biopsy needle 66 according to a target insertion angle. The biopsy needle 66 thus disposed by the disposition mechanism 62 is made to puncture a breast W, and the target is collected using the biopsy needle 66, that is, a biopsy is performed. In the present embodiment, the “insertion angle” of the biopsy needle 66 refers to an angle formed between a line perpendicular to the imaging surface 40A of the imaging table 40 and the biopsy needle 66 (see an angle θ in FIGS. 5A and 5B).

Further, as shown in FIG. 2, the medical imaging apparatus 10 of the present embodiment comprises the ultrasound probe 70 on which an acoustic matching body 71 is mounted, and a moving mechanism 72. Note that the target in the present embodiment is an example of a region of interest of the present disclosure.

The ultrasound probe 70 is used to scan the breast with ultrasound to acquire an ultrasound image of the breast. The ultrasound probe 70 comprises a plurality of ultrasound transducers (not shown) which are one-dimensionally or two-dimensionally arranged. Each of the ultrasound transducers transmits ultrasonic waves based on an applied drive signal, receives ultrasound echoes, and outputs a received signal. The ultrasound probe 70 is provided with the acoustic matching body 71 so as to cover at least the ultrasound transmission surface in contact with the breast. The acoustic matching body 71 is made of a member having an acoustic impedance close to that of a living body (breast), and in the present embodiment, is in the form of a gel or a sheet. As such an acoustic matching body 71, an acoustic coupler or the like can be used. Specific examples thereof include an acoustic coupler of a type in which a polyurethane gel pad is fixed to the ultrasound probe 70 with a fixing jig.

The moving mechanism 72 has a function of moving the ultrasound probe 70 in the front-rear, left-right, and up-down directions. As shown in FIG. 4, the moving mechanism 72 includes one moving rail 72X and two moving rails 72Y. The moving rail 72X is a rail extending in the left-right direction, and the ultrasound probe 70 is attached to the moving rail 72X. The moving mechanism 72 changes the position of the ultrasound probe 70 in the left-right direction by moving the ultrasound probe 70 along the moving rail 72X.

Further, the moving rail 72Y is a rail provided at each of both ends of the imaging surface 40A of the imaging table 40 and extending in the front-rear direction. The moving mechanism 72 changes the position of the ultrasound probe 70 in the front-rear direction by moving the moving rail 72X to which the ultrasound probe 70 is attached in the front-rear direction.

Moreover, the moving mechanism 72 can also change the height of the ultrasound probe 70 by moving the moving rails 72X and 72Y in the up-down direction.

Further, in the present embodiment, as shown in FIG. 4, the moving mechanism 72 allows the ultrasound probe 70 to be rotated (rotation angle q) to change the direction in which it contacts the breast. For example, as shown in FIG. 5A, in a case in which the ultrasound probe 70 is rotated to dispose transducers in a direction that intersects a major axis direction of the biopsy needle 66, ultrasound images can be captured in a minor axis direction of the biopsy needle 66. In this case, as shown in FIG. 5A, as an example, an ultrasound image 80 obtained by the ultrasound probe 70 includes a cross-sectional image 81 of the biopsy needle 66 in the minor axis direction and a target image 82 of the target.

On the other hand, in a case in which the ultrasound probe 70 is rotated to dispose transducers in a direction that intersects the minor axis direction of the biopsy needle 66, ultrasound images can be captured in the major axis direction of the biopsy needle 66. In this case, as shown in FIG. 5B, as an example, the ultrasound image 80 obtained by the ultrasound probe 70 includes a cross-sectional image 81 of the biopsy needle 66 in the major axis direction and the target image 82 of the target.

In this manner, by rotating the ultrasound probe 70, the cross-sectional image 81 of the biopsy needle 66 shown in the ultrasound image can be switched between a cross-sectional image in the minor axis direction and a cross-sectional image in the major axis direction. In this manner, by switching the state of the cross-sectional image of the biopsy needle 66 shown in the ultrasound image 80, it is possible to easily observe the entry trajectory of the biopsy needle 66 and the position of the needle tip from different angles. Moreover, the positional relationship between the biopsy needle 66 and a target T can be easily understood spatially.

On the other hand, the console 12 of the present embodiment has a function of controlling the medical imaging apparatus 10 using an imaging order and various types of information acquired from a radiology information system (RIS) 5 or the like through a wireless communication local area network (LAN), instructions input by the user using an operation unit 56 or the like, and the like. The console 12 is also connected to an image storage system 19 through wireless communication or wired communication, and has a function of transmitting radiation images and ultrasound images obtained by the medical imaging apparatus 10 to the image storage system 19 such as picture archiving and communication systems (PACS) and storing the images. Note that the console 12 of the present embodiment is an example of a guide device according to the present disclosure.

Furthermore, the configuration of the medical imaging apparatus 10 and the console 12 will be described with reference to FIG. 6. FIG. 6 is a block diagram showing an example of the configuration of the medical imaging apparatus 10 and the console 12.

As shown in FIG. 6, the medical imaging apparatus 10 further comprises a controller 20, a storage unit 22, an I/F unit 24, and an operation unit 26. The controller 20, the storage unit 22, the I/F unit 24, the operation unit 26, the radiation detector 30, the compression member driving unit 32, the radiation emitting unit 36, the radiation source moving unit 37, the biopsy unit 60, the ultrasound probe 70, and the moving mechanism 72 are connected to each other through a bus 29 such as a system bus or a control bus, such that they can exchange various types of information with each other.

The controller 20 of the present embodiment controls the overall operation of the medical imaging apparatus 10. The controller 20 comprises a CPU 20A, a ROM 20B, and a RAM 20C. For example, various programs executed by the CPU 20A, such as an imaging processing program 21 executed in the case of controlling imaging, are stored in the ROM 20B in advance. The RAM 20C transitorily stores various types of data.

The storage unit 22 stores, for example, a radiation image captured using the radiation detector 30, an ultrasound image captured using the ultrasound probe 70, and various other types of information. Specific examples of the storage unit 22 include an HDD, an SSD, and the like.

The operation unit 26 is used by the user to input, for example, instructions or various types of information regarding imaging and biopsy. Note that, the operation unit 26 is not particularly limited, and examples of the operation unit 26 include various switches, a touch panel, a touch pen, a mouse, and the like.

The I/F unit 24 communicates radiation images, ultrasound images, and various types of information with the console 12 through wireless communication or wired communication.

On the other hand, as shown in FIG. 6, the console 12 comprises a controller 50, a storage unit 52, an I/F unit 54, an operation unit 56, and a display unit 58. The controller 50, the storage unit 52, the I/F unit 54, the operation unit 56, and the display unit 58 are connected to each other through a bus 59 such as a system bus or a control bus, such that they can exchange various types of information with each other.

The controller 50 of the present embodiment controls the overall operation of the console 12. The controller 50 comprises a CPU 50A, a ROM 50B, and a RAM 50C. For example, various programs executed by the CPU 50A, such as a biopsy processing program 51 executed in the case of controlling a biopsy, are stored in the ROM 50B in advance. The RAM 50C transitorily stores various types of data.

The storage unit 52 stores, for example, radiation images and ultrasound images captured by the medical imaging apparatus 10 and various other types of information. Specific examples of the storage unit 52 include an HDD, an SSD, and the like.

The operation unit 56 is used by the user to input, for example, instructions or various types of information regarding imaging and biopsy. Note that, the operation unit 56 is not particularly limited, and examples of the operation unit 56 include various switches, a touch panel, a touch pen, a mouse, and the like. The display unit 58 displays various types of information. Note that, the operation unit 56 and the display unit 58 may be integrated into a touch panel display.

The I/F unit 54 communicates radiation images, ultrasound images, and various types of information with each of the RIS 5, the medical imaging apparatus 10, and the image storage system 19 through wireless communication or wired communication.

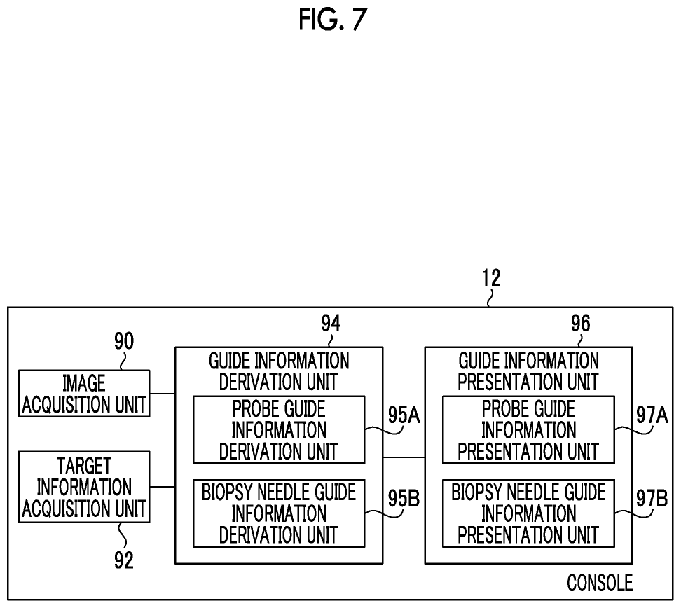

Further, FIG. 7 shows a functional block diagram showing an example of a function of the console 12. The console 12 comprises an image acquisition unit 90, a target information acquisition unit 92, a guide information derivation unit 94, and a guide information presentation unit 96. As an example, the console 12 of the present embodiment functions as the image acquisition unit 90, the target information acquisition unit 92, the guide information derivation unit 94, and the guide information presentation unit 96.

The image acquisition unit 90 has a function of acquiring a radiation image or an ultrasound image related to a biopsy. In the present embodiment, scout imaging in which the breast is imaged in two directions by varying an irradiation angle of the radiation such that a target region of the breast to be subjected to the biopsy is included before the puncture is performed to acquire a scout image. The scout image is an image viewed from different viewpoints in order to check the position to be pathologically examined. The user designates a target to be subjected to a biopsy on the scout image. Therefore, the image acquisition unit 90 acquires the scout image. Further, the image acquisition unit 90 acquires an ultrasound image captured using the ultrasound probe 70. The image acquisition unit 90 outputs these acquired images to the guide information derivation unit 94. The target to be subjected to the biopsy is not particularly limited, and representative examples thereof include a lesion portion including at least one of calcification, a tumor, a spicula, or the like.

The target information acquisition unit 92 has a function of acquiring information regarding a target designated by the user as described above. Specifically, the target information acquisition unit 92 acquires information indicating the position of the target designated on the scout image as target information. The target information acquisition unit 92 outputs the acquired target information to the guide information derivation unit 94.

The guide information derivation unit 94 includes a probe guide information derivation unit 95A and a biopsy needle guide information derivation unit 95B. The biopsy needle guide information derivation unit 95B has a function of deriving a target insertion position and a target insertion angle for inserting the biopsy needle 66 into the breast. As an example, the biopsy needle guide information derivation unit 95B of the present embodiment derives a target insertion position and a target insertion angle based on the position of the target (three-dimensional position) designated by the user.

The probe guide information derivation unit 95A has a function of deriving a target scanning position that is a target scanning position of the ultrasound probe 70. As an example, the probe guide information derivation unit 95A of the present embodiment derives a target scanning position of the ultrasound probe 70 based on the target insertion position and target insertion angle of the biopsy needle 66, and the position of the opening portion 34B of the bottom portion 34A of the compression member 34 in a state in which the breast is compressed. Specifically, the probe guide information derivation unit 95A derives a final destination of the biopsy needle 66 from the target insertion angle and the target insertion position of the biopsy needle 66. The scanning position of the ultrasound probe 70 is preferably above the final destination. The probe guide information derivation unit 95A derives a position within the opening portion 34B of the compression member 34 that is closest to the final destination, where the acoustic matching body 71 provided on the ultrasound probe 70 can maintain a non-contact state with the biopsy needle 66 even in a case in which the ultrasound probe 70 is rotated as described above. Even in a case in which the ultrasound probe 70 is rotated, in a case in which a position where the acoustic matching body 71 provided on the ultrasound probe 70 is in a non-contact state with the biopsy needle 66 is outside the opening portion 34B of the compression member 34, it is preferable to notify the user of that effect in advance. Alternatively, in a case in which the biopsy needle 66 and the ultrasound probe 70 are inserted into the opening portion 34B of the compression member 34, and in a case in which the acoustic matching body 71 comes into contact with the biopsy needle 66 as the ultrasound probe 70 is rotated, it is preferable to warn the user of that effect. The guide information derivation unit 94 outputs, to the guide information presentation unit, the target scanning position of the ultrasound probe 70 and the target insertion position and the target insertion angle of the biopsy needle 66 which are derived in this manner.

The guide information presentation unit 96 includes a probe guide information presentation unit 97A and a biopsy needle guide information presentation unit 97B. The biopsy needle guide information presentation unit 97B outputs, to the medical imaging apparatus 10, biopsy needle guide information for moving the biopsy needle 66 to the target insertion position and tilting the biopsy needle 66 to the target insertion angle. Thereby, in the medical imaging apparatus 10, the biopsy needle 66 is moved by the disposition mechanism 62 based on the biopsy needle guide information.

Further, the probe guide information presentation unit 97A outputs, to the medical imaging apparatus 10, probe guide information for moving the ultrasound probe 70 to the target scanning position. Thereby, in the medical imaging apparatus 10, the ultrasound probe 70 is moved by the moving mechanism 72 based on the probe guide information.

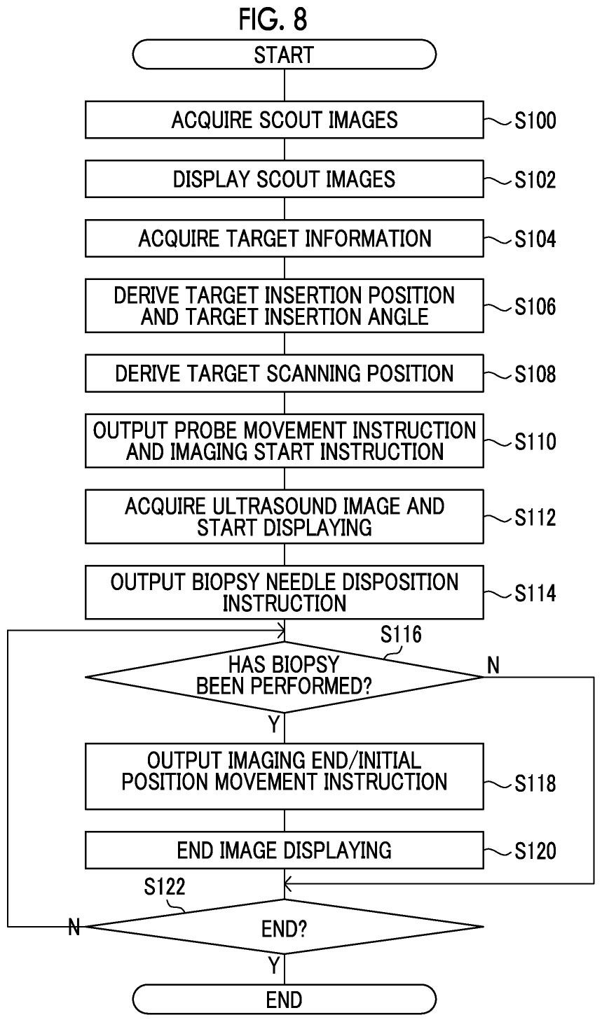

Next, the operation of the console 12 of the present embodiment in the biopsy will be described with reference to the drawings, and FIG. 8 is a flowchart showing an example of the flow of biopsy processing executed in the console 12. Note that the biopsy processing of the present embodiment is an example of guide processing according to the present disclosure.

In Step S100 of FIG. 8, the image acquisition unit 90 acquires scout images, that is, two radiation images captured at different irradiation angles of radiation, as described above.

Then, in Step S102, the image acquisition unit 90 causes the display unit 58 to display the scout images acquired in Step S100. As described above, the user designates a target on the displayed scout image. Therefore, in the next Step S104, the target information acquisition unit 92 outputs target information indicating the position of the target designated by the user.

In the next Step S106, the biopsy needle guide information derivation unit 95B of the guide information derivation unit 94 derives the target insertion position and the target insertion angle of the biopsy needle 66, as described above.

In the next Step S108, the probe guide information derivation unit 95A of the guide information derivation unit 94 derives the target scanning position of the ultrasound probe 70, as described above.

In the next Step S110, the probe guide information presentation unit 97A of the guide information presentation unit 96 outputs probe guide information for moving the ultrasound probe 70 to the target scanning position, and outputs an instruction to start capturing an ultrasound image, to the medical imaging apparatus 10.

Thereby, in the medical imaging apparatus 10, the moving mechanism 72 moves the ultrasound probe 70 to the target scanning position as described above. Further, the ultrasound probe 70 inserted into the opening portion 34B of the compression member 34 is brought into contact with the breast via the acoustic matching body 71 to start capturing an ultrasound image. The medical imaging apparatus 10 sequentially outputs ultrasound images obtained by imaging to the console 12. Note that, as described above, in the present embodiment, the ultrasound probe 70 can be rotated. The rotation of the ultrasound probe 70 is appropriately performed at the timing instructed by the user using the operation unit 56 of the console 12.

In the next Step S112, the image acquisition unit 90 acquires the ultrasound image captured as described above from the medical imaging apparatus 10 and causes the display unit 58 to display the ultrasound image. The user can determine whether or not the biopsy needle 66 has been appropriately inserted based on the displayed ultrasound image.

In the next Step S114, the biopsy needle guide information presentation unit 97B outputs, to the medical imaging apparatus 10, biopsy needle guide information for moving the biopsy needle 66 to the target insertion position and tilting the biopsy needle 66 to the target insertion angle.

Accordingly, in the medical imaging apparatus 10, as described above, the disposition mechanism 62 disposes the biopsy needle 66 such that the biopsy needle 66 is tilted to the target insertion angle and the position of the distal end of the biopsy needle 66 is at the target insertion position. The user punctures the breast with the biopsy needle 66 disposed in this manner and performs a biopsy to collect the target tissue.

Then, in the next Step S116, the guide information presentation unit 96 determines whether or not a biopsy has been performed. In a case in which a biopsy has not been performed, the determination in Step S116 is negative, and the process proceeds to Step S122. On the other hand, in a case in which a biopsy has been performed, the determination in Step S116 is affirmative, and the process proceeds to Step S118.

In Step S118, the guide information presentation unit 96 outputs an imaging end instruction to end capturing an ultrasound image and a movement instruction to move the position of the biopsy needle 66 and the position of the ultrasound probe 70 to the position of the initial state. Thereby, in the medical imaging apparatus 10, the driving of the ultrasound probe 70 is stopped, and the moving mechanism 72 moves the ultrasound probe 70 to the initial position. Furthermore, in the medical imaging apparatus 10, the disposition mechanism 62 moves the biopsy needle 66 to the initial position. In the nest Step S120, the image acquisition unit 90 ends displaying the ultrasound image.

In the next Step S122, it is determined whether or not to end the biopsy processing. In a case in which the biopsy processing is not ended yet, the determination in Step S122 is negative, the process returns to Step S116, and the processes of Steps S116 to S120 are repeated.

On the other hand, in the embodiment, in a case in which the biopsy is appropriately performed and there is no need to redo the biopsy, or in a case in which the biopsy is to be canceled, the determination in Step S122 is affirmative, and the biopsy processing shown in FIG. 8 ends.

In the above embodiment, the position of the target is specified based on the scout image. However, the method for specifying the position of the target is not limited to a method using a scout image, but it is preferable that the three-dimensional position of the target can be specified. Such a method may be, for example, a radiation image other than a scout image, such as a tomographic image, or an ultrasound image captured in advance.

Further, in the present embodiment, the aspect in which the target information acquisition unit 92 acquires information indicating the position of the target designated by the user with respect to the scout image has been described. However, the target information acquisition unit 92 may be configured to acquire target information by detecting a target from an image. In this case, for example, the target information acquisition unit 92 may detect the target from the radiation image or the ultrasound image using computer-aided detection (CAD). Although there are no particular limitations on which images are used to acquire what type of target information, considering the respective features of radiation images and ultrasound images, it is preferable to use radiation images for detecting calcification and to use ultrasound images for detecting tumors.

### Second Embodiment

In the first embodiment, the aspect in which the biopsy needle 66 is automatically moved and the ultrasound probe 70 is automatically moved has been described. However, at least one of the biopsy needle 66 or the ultrasound probe 70 may be configured to be moved by the user. For example, in a case in which a radiology technician moves the ultrasound probe 70, the medical imaging apparatus 10 may be configured by combining a mammography apparatus capable of performing a biopsy and an ultrasonography apparatus separate from the mammography apparatus instead of the medical imaging apparatus 10. In this case, the guide information presentation unit 96 of the console 12 may cause the display unit 58 to display probe guide information indicating the target scanning position of the ultrasound probe 70.

### Third Embodiment

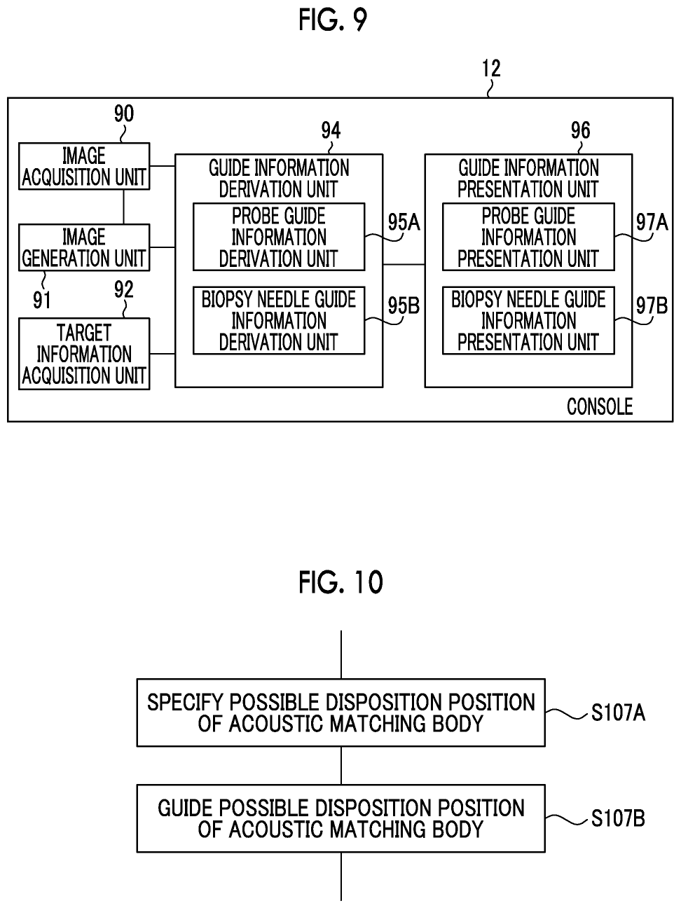

Other forms of guide information will be described. An ultrasound image such as a three-dimensional ultrasound image generated from an ultrasound image captured using the ultrasound probe 70 during the biopsy processing may be used as the guide information. In this case, as shown in FIG. 9, the console 12 includes an image generation unit 91 and generates a three-dimensional ultrasound image from the plurality of ultrasound images acquired by the image acquisition unit 90. In this case, it is preferable that the image is a three-dimensional ultrasound image of a cross section along the target insertion position and target insertion angle of the biopsy needle 66. Note that the image generation unit 91 may be an image processing apparatus separate from the console 12, and the console 12 may be configured to acquire a three-dimensional ultrasound image generated by the image processing apparatus. The image generation unit 91 of the present embodiment is an example of an image processing apparatus of the present disclosure.

In addition, it is preferable that the image generation unit 91 acquires the current insertion position and insertion angle of the biopsy needle 66, derives the remaining distance or the like to the target from the current state of the biopsy needle 66, adds this information to the above-mentioned three-dimensional ultrasound image, and presents the information to the user. For example, as described above, the remaining distance to the target may be displayed in the ultrasound image of a cross section corresponding to the insertion angle.

### Fourth Embodiment

In each of the above embodiments, the aspect in which the acoustic matching body 71 is mounted on the ultrasound probe 70, in other words, the aspect in which the ultrasound probe 70 and the acoustic matching body 71 are integrated, has been described, but the acoustic matching body 71 may be separate from the ultrasound probe 70.

In addition, since it is preferable that the acoustic matching body 71 does not spread beyond the range where the user has disposed the acoustic matching body 71, for example, a liquid acoustic matching body 71 with low viscosity is not preferable; for example, a gel-like one is preferable, and an echo gel pad or the like is preferable.

In this way, in a case in which the acoustic matching body 71 separate from the ultrasound probe 70 is provided, the user disposes the acoustic matching body 71. Therefore, it is preferable that the console 12 guides the user to the disposition position of the acoustic matching body 71 that does not come into contact with the biopsy needle 66.

In the biopsy processing executed by the console 12 in this case, processes of Steps S107A and S107B shown in FIG. 10 are performed between Steps S106 and S108 of the above-mentioned biopsy processing (see FIG. 8).

In Step S107A of FIG. 10, the guide information derivation unit 94 derives, as a possible disposition position of the acoustic matching body 71, a region where the biopsy needle 66 and the ultrasound probe 70 do not come into contact even in a case in which the biopsy needle 66 is inserted into the breast W, based on the target insertion position and the target insertion angle of the biopsy needle 66.

In the next Step S107B, the guide information presentation unit 96 presents guide information for guiding the possible disposition position of the acoustic matching body 71 derived in Step S107A.

Note that the method of presenting guide information is not limited. For example, an image showing the disposition position of the breast, such as a radiation image obtained by imaging the breast, may be displayed on the display unit 58, and information indicating the possible disposition position of the acoustic matching body 71 may be displayed on the image. For example, in a case in which the medical imaging apparatus 10 includes a visible light source, a collimator, and the like for showing the user a small irradiation range of the irradiation beam by emitting visible light, these may be used to present the possible disposition position of the acoustic matching body 71 using visible light.

In the present embodiment, the position of the acoustic matching body 71 actually disposed by the user is detected, and in a case in which the acoustic matching body 71 is disposed beyond the possible disposition position of the acoustic matching body 71, it is preferable to give a warning to the user. For example, it is sufficient to perform image analysis on a captured image obtained by imaging a state of the acoustic matching body 71 disposed on the breast with a camera, specify the actual position of the acoustic matching body 71, and compare the actual disposition position with the possible disposition position of the acoustic matching body 71.

As described above, according to the biopsy system 2 of each of the above embodiments, the biopsy needle 66 and the ultrasound probe 70 can be inserted into the opening portion 34B of the compression member 34 and the distance between the ultrasound probe 70 and the target can be shortened. Therefore, the image quality of the ultrasound image captured by the ultrasound probe 70 can be made high. Further, since the acoustic matching body 71 and the biopsy needle 66 can be made non-contact, it is possible to suppress the biopsy needle 66 from being contaminated by the acoustic matching body 71. Therefore, according to the biopsy system 2 of each of the above embodiments, a biopsy of a breast can be performed with reference to a high-quality ultrasound image while preventing contamination of the biopsy needle 66.

Further, in the above-described embodiment, for example, as hardware structures of processing units that execute various kinds of processing, such as the image acquisition unit 90, the target information acquisition unit 92, the guide information derivation unit 94, and the guide information presentation unit 96, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (program).

One processing unit may be configured by one of the various processors, or may be configured by a combination of the same or different types of two or more processors (for example, a combination of a plurality of FPGAs or a combination of the CPU and the FPGA). In addition, a plurality of processing units may be configured by one processor.

As an example in which a plurality of processing units are configured by one processor, first, a form in which one processor is configured by a combination of one or more CPUs and software as typified by a computer, such as a client or a server, and this processor functions as a plurality of processing units can be mentioned. Second, as represented by a system-on-chip (SoC) or the like, a form of using a processor for realizing the function of the entire system including a plurality of processing units with one integrated circuit (IC) chip can be mentioned. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In each of the above embodiments, the biopsy processing program 51 has been described as being stored (installed) in advance in the ROM 50B of the console 12; however, the present disclosure is not limited thereto. The biopsy processing program 51 may be provided in a form recorded in a recording medium such as a compact disc read-only memory (CD-ROM), a digital versatile disc read-only memory (DVD-ROM), and a universal serial bus (USB) memory. In addition, the biopsy processing program 51 may be configured to be downloaded from an external device via a network.

In addition, the configurations and operations of the biopsy system 2, the medical imaging apparatus 10, the console 12, and the like described in each of the above-described embodiments are merely examples and it goes without saying that they can be changed according to the situation without departing from the gist of the present invention. Furthermore, it goes without saying that the above-described embodiments may be combined as appropriate.

Regarding the above embodiments, the following supplementary notes are further disclosed.

### Supplementary Note 1

A biopsy system that performs a biopsy of a breast while capturing an ultrasound image with an ultrasonography apparatus, the biopsy system comprising:
a compression member that has an opening portion into which a biopsy needle and an ultrasound probe used for capturing the ultrasound image are inserted and that compresses the breast;
at least one processor configured to perform guide processing for guiding a target insertion position and a target insertion angle of the biopsy needle inserted into the breast and a target scanning position of the ultrasound probe; and
an acoustic matching body that is provided between the ultrasound probe and the breast in a non-contact state with the biopsy needle.

### Supplementary Note 2

The biopsy system according to Supplementary Note 1, in which the acoustic matching body is mounted on the ultrasound probe.

### Supplementary Note 3

The biopsy system according to Supplementary Note 1 or 2, in which the processor is configured to:
acquire a current insertion position and a current insertion angle of the biopsy needle inserted into the breast;
acquire a current scanning position of the ultrasound probe; and
perform the guide processing based on the current insertion position and the current insertion angle of the biopsy needle, the current scanning position of the ultrasound probe, a current position of a distal end of the biopsy needle, the target insertion position and the target insertion angle of the biopsy needle, and the target scanning position of the ultrasound probe.

### Supplementary Note 4

The biopsy system according to any one of Supplementary Notes 1 to 3,
in which the processor is configured to, as the guide processing:
   acquire at least one of a radiation image of the breast or the ultrasound image captured by the ultrasonography apparatus;
   derive the target insertion position and the target insertion angle of the biopsy needle based on a region of interest in the radiation image of the breast or a region of interest in the ultrasound image of the breast, and present the derived target insertion position and target insertion angle of the biopsy needle; and
   derive the target scanning position of the ultrasound probe that is in a non-contact position with the biopsy needle based on the derived target insertion position and target insertion angle of the biopsy needle, and present the derived target scanning position of the ultrasound probe.

### Supplementary Note 5

The biopsy system according to Supplementary Note 4,
in which the processor is configured to:
   in a case in which a target is calcification, derive the target insertion position and the target insertion angle of the biopsy needle based on the region of interest in the radiation image; and
   in a case in which the target is a tumor, derive the target insertion position and a target insertion angle of the biopsy needle based on the region of interest in the ultrasound image.

### Supplementary Note 6

The biopsy system according to any one of Supplementary Notes 1 to 5, further comprising:
a moving mechanism that moves the ultrasound probe,
in which the processor is configured to present the target scanning position of the ultrasound probe to the moving mechanism, and
the moving mechanism moves the ultrasound probe to the target scanning position.

### Supplementary Note 7

The biopsy system according to Supplementary Note 6,
in which the moving mechanism enables capturing of ultrasound images in a major axis direction and a minor axis direction of the biopsy needle by rotating the ultrasound probe with respect to the biopsy needle.

### Supplementary Note 8

The biopsy system according to any one of Supplementary Notes 1 to 7,
in which the processor is configured to perform guide processing for guiding a scanning position of the ultrasound probe to a position where a final destination of the biopsy needle based on the derived target insertion position and target insertion angle of the biopsy needle is able to be imaged.

### Supplementary Note 9

The biopsy system according to any one of Supplementary Notes 1 to 8, further comprising:
a disposition mechanism for disposing the biopsy needle,
in which the processor is configured to present the target insertion position and the target insertion angle of the biopsy needle to the disposition mechanism, and
the disposition mechanism disposes the biopsy needle according to the target scanning position and the target insertion angle.

### Supplementary Note 10

The biopsy system according to any one of Supplementary Notes 1 to 9,
in which the processor is configured to:
determine whether or not there is a likelihood that the acoustic matching body and the biopsy needle come into contact with each other; and
give a warning in a case in which there is a likelihood of contact.

### Supplementary Note 11

The biopsy system according to any one of Supplementary Notes 1 to 10, further comprising:
an image processing apparatus configured to:
   acquire a plurality of ultrasound images captured by the ultrasonography apparatus; and
   generate a three-dimensional ultrasound image from the plurality of ultrasound images.

### Supplementary Note 12

The biopsy system according to any one of Supplementary Notes 1 to 11,
in which the acoustic matching body is in a form of a gel, and
the processor is configured to:
   acquire at least one of a radiation image of the breast or the ultrasound image captured by the ultrasonography apparatus;
   derive the target insertion position and the target insertion angle of the biopsy needle based on a region of interest in the radiation image of the breast or a region of interest in the ultrasound image of the breast; and
   further perform guide processing for guiding a disposition position of the acoustic matching body to a disposition position where the acoustic matching body does not come into contact with the biopsy needle based on the derived target insertion position and target insertion angle of the biopsy needle.

### Supplementary Note 13

A guide device used in a biopsy system that performs a biopsy of a breast while capturing an ultrasound image with an ultrasonography apparatus, the guide device comprising:
at least one processor,
in which the processor is configured to perform guide processing for guiding a target insertion position and a target insertion angle of a biopsy needle inserted into the breast and a target scanning position of an ultrasound probe.

### Supplementary Note 14

A biopsy method using a biopsy system that performs a biopsy of a breast while capturing an ultrasound image with an ultrasonography apparatus, the biopsy method comprising:
performing guide processing for guiding, by a processor, in a state in which the breast is compressed by a compression member having an opening portion into which a biopsy needle and an ultrasound probe used for capturing the ultrasound image are inserted, and an acoustic matching body is provided between the ultrasound probe and the breast in a non-contact state with the biopsy needle, a target insertion position and a target insertion angle of the biopsy needle inserted into the breast and a target scanning position of the ultrasound probe.

What is claimed is:

1. A biopsy system that performs a biopsy of a breast while capturing an ultrasound image with an ultrasonography apparatus, the biopsy system comprising:
a compression member that has an opening portion into which a biopsy needle and an ultrasound probe used for capturing the ultrasound image are inserted and that compresses the breast;
at least one processor configured to perform guide processing for guiding a target insertion position and a target insertion angle of the biopsy needle inserted into the breast and a target scanning position of the ultrasound probe; and
an acoustic matching body that is provided between the ultrasound probe and the breast in a non-contact state with the biopsy needle.

2. The biopsy system according to claim 1,
wherein the acoustic matching body is mounted on the ultrasound probe.

3. The biopsy system according to claim 1,
wherein the processor is configured to:
acquire a current insertion position and a current insertion angle of the biopsy needle inserted into the breast;
acquire a current scanning position of the ultrasound probe; and
perform the guide processing based on the current insertion position and the current insertion angle of the biopsy needle, the current scanning position of the ultrasound probe, a current position of a distal end of the biopsy needle, the target insertion position and the target insertion angle of the biopsy needle, and the target scanning position of the ultrasound probe.

4. The biopsy system according to claim 1,
wherein the processor is configured to, as the guide processing:
acquire at least one of a radiation image of the breast or the ultrasound image captured by the ultrasonography apparatus;
derive the target insertion position and the target insertion angle of the biopsy needle based on a region of interest in the radiation image of the breast or a region of interest in the ultrasound image of the breast, and present the derived target insertion position and target insertion angle of the biopsy needle; and
derive the target scanning position of the ultrasound probe that is in a non-contact position with the biopsy needle based on the derived target insertion position and target insertion angle of the biopsy needle, and present the derived target scanning position of the ultrasound probe.

5. The biopsy system according to claim 4,
wherein the processor is configured to:
in a case in which a target is calcification, derive the target insertion position and the target insertion angle of the biopsy needle based on the region of interest in the radiation image; and
in a case in which the target is a tumor, derive the target insertion position and the target insertion angle of the biopsy needle based on the region of interest in the ultrasound image.

6. The biopsy system according to claim 1, further comprising:
a moving mechanism that moves the ultrasound probe,
wherein the processor is configured to present the target scanning position of the ultrasound probe to the moving mechanism, and
the moving mechanism moves the ultrasound probe to the target scanning position.

7. The biopsy system according to claim 6,
wherein the moving mechanism enables capturing of ultrasound images in a major axis direction and a minor axis direction of the biopsy needle by rotating the ultrasound probe with respect to the biopsy needle.

8. The biopsy system according to claim 1,
wherein the processor is configured to perform guide processing for guiding a scanning position of the ultrasound probe to a position where a final destination of the biopsy needle based on the derived target insertion position and target insertion angle of the biopsy needle is able to be imaged.

9. The biopsy system according to claim 1, further comprising:
a disposition mechanism for disposing the biopsy needle,
wherein the processor is configured to:
present the target insertion position and the target insertion angle of the biopsy needle to the disposition mechanism; and
the disposition mechanism disposes the biopsy needle according to the target scanning position and the target insertion angle.

10. The biopsy system according to claim 1,
wherein the processor is configured to:
determine whether or not there is a likelihood that the acoustic matching body and the biopsy needle come into contact with each other; and
give a warning in a case in which there is a likelihood of contact.

11. The biopsy system according to claim 1, further comprising:
an image processing apparatus configured to:
acquire a plurality of ultrasound images captured by the ultrasonography apparatus; and
generate a three-dimensional ultrasound image from the plurality of ultrasound images.

12. The biopsy system according to claim 1,
wherein the acoustic matching body is in a form of a gel, and
the processor is configured to:
acquire at least one of a radiation image of the breast or the ultrasound image captured by the ultrasonography apparatus;
derive the target insertion position and the target insertion angle of the biopsy needle based on a region of interest in the radiation image of the breast or a region of interest in the ultrasound image of the breast; and
further perform guide processing for guiding a disposition position of the acoustic matching body to a disposition position where the acoustic matching body does not come into contact with the biopsy needle based on the derived target insertion position and target insertion angle of the biopsy needle.

13. A guide device used in a biopsy system that performs a biopsy of a breast while capturing an ultrasound image with an ultrasonography apparatus, the guide device comprising:
at least one processor,
wherein the processor is configured to perform guide processing for guiding a target insertion position and a target insertion angle of a biopsy needle inserted into the breast and a target scanning position of an ultrasound probe.

14. A biopsy method using a biopsy system that performs a biopsy of a breast while capturing an ultrasound image with an ultrasonography apparatus, the biopsy method comprising:
performing guide processing for guiding, by a processor, in a state in which the breast is compressed by a compression member having an opening portion into which a biopsy needle and an ultrasound probe used for capturing the ultrasound image are inserted, and an acoustic matching body is provided between the ultrasound probe and the breast in a non-contact state with the biopsy needle, a target insertion position and a target insertion angle of the biopsy needle inserted into the breast and a target scanning position of the ultrasound probe.
